(19) United States
(12) Patent Application Publication
(10) Pub. No.: US 2007/0101452 A1
(43) Pub. Date: May 3, 2007

(54) Method for creating fusion protein and conditional alleles

(76) Inventors: Scott E. Fraser, La Canada, CA (US); Sean Megason, Los Angeles, CA (US)

Correspondence Address: LADAS & PARRY;Suite 2100, 5670 Wilshire Boulevard, Los Angeles, CA, 90036-5679, US

(21) Appl. No.: 11/546,517

(22) Filed: Oct. 10, 2006

Related U.S. Application Data
(60) Provisional application No. 60/726,375, filed on Oct. 13, 2005.

(51) Int. Cl. A01K 67/027; C12N 5/06; C12N 15/86

(52) U.S. Cl. 800/18; 435/455; 435/354; 435/456

(57) ABSTRACT

Gene trapping provides insertional mutagenesis strategies for monitoring the expression and localization of endogenous proteins. In accordance with the disclosure herein, a gene trapping vector and a method of fabrication and use are disclosed which provide for an efficient means of trapping and analyzing a gene of interest.

### CROSS REFERENCE TO RELATED APPLICATIONS

This application claims priority to U.S. Provisional Ser. No. 60/726,375 for “Efficient Method for Creating fusion Protein and Conditional Alleles” filed on Oct. 13, 2005 which is incorporated herein by reference in its entirety.

### STATEMENT OF GOVERNMENT SUPPORT

The invention described herein was made under a grant from the National Institute of Health, Grant No. R01HD043897. The U.S. government may have certain rights in the invention.

### BACKGROUND

1. Field

The present disclosure relates to a nucleic acid vector and methods for creating fusion proteins and conditional alleles.

2. Description of Related Art

Methods of gene trapping provide insertional mutagenesis strategies for monitoring the expression and localization of endogenous proteins. Such methods aid in the elucidation of gene function and diseases associated with gene mutations.

### SUMMARY

According to a first embodiment of the present disclosure, a nucleic acid vector is provided comprising a nucleic acid backbone sequence; a splice acceptor sequence; a splice donor sequence; a first recombination site and a second recombination site forming a first recombination site pair capable of being recognized by a first recombinase, wherein the first and second recombination sites are in opposite orientations; a third recombination site and a fourth recombination site forming a second recombination site pair capable of being recognized by the first recombinase or a second recombinase, wherein the first and second recombination sites are in opposite orientations, and wherein the second recombination site pair flanks the second recombination site, but does not flank the first recombination site, and a polyadenylation sequence.

According to a second embodiment of the present disclosure, a method of creating a conditional allele is provided, the method comprising: introducing into a cell, a nucleic acid vector; said nucleic acid vector comprising a nucleic acid backbone sequence, a splice acceptor sequence, a splice donor sequence, a first recombination site and a second recombination site forming a first recombination site pair capable of being recognized by a first recombinase, wherein the first and second recombination sites are in opposite orientations, a third recombination site and a fourth recombination site forming a second recombination site pair capable of being recognized by the first recombinase or a second recombinase, wherein the first and second recombination sites are in opposite orientations, and wherein the second recombination site pair flanks the second recombination site, but does not flank the first recombination site, and a polyadenylation sequence; the method further comprising introducing into the cell a first recombinase creating a first recombination event; and introducing into the cell a second recombinase creating a second recombination event.

According to a third embodiment of the present disclosure, a method of creating a conditional allele is provided, the method comprising: introducing into a cell, a nucleic acid vector; said nucleic acid vector comprising a nucleic acid backbone sequence, a splice acceptor sequence, a splice donor sequence, a first recombination site and a second recombination site forming a first recombination site pair capable of being recognized by a first recombinase, wherein the first and second recombination sites are in opposite orientations, a third recombination site and a fourth recombination site forming a second recombination site pair capable of being recognized by the first recombinase or a second recombinase, wherein the first and second recombination sites are in opposite orientations, and wherein the second recombination site pair flanks the second recombination site, but does not flank the first recombination site, a first detectable marker sequence, and a polyadenylation sequence; the method further comprising introducing into the cell a first recombinase creating a first recombination event; and introducing into the cell a second recombinase creating a second recombination event; and selecting for the presence of a protein expressed from the first detectable marker.

According to a fourth embodiment of the present disclosure, a method of creating a conditional allele is provided, the method comprising: introducing into a cell, a nucleic acid vector; said nucleic acid vector comprising a nucleic acid backbone sequence, a splice acceptor sequence, a splice donor sequence, a first recombination site and a second recombination site forming a first recombination site pair capable of being recognized by a first recombinase, wherein the first and second recombination sites are in opposite orientations, a third recombination site and a fourth recombination site forming a second recombination site pair capable of being recognized by the first recombinase or a second recombinase, wherein the first and second recombination sites are in opposite orientations, and wherein the second recombination site pair flanks the second recombination site, but does not flank the first recombination site, a first detectable marker sequence, a second detectable marker and a polyadenylation sequence; the method further comprising introducing into the cell a first recombinase creating a first recombination event; and introducing into the cell a second recombinase creating a second recombination event; selecting for the presence of a protein expressed from the first detectable marker, and selecting for the presence of a protein expressed from the second detectable marker.

Additional embodiments are presented in the enclosed claims and /or in the detailed description.

### BRIEF DESCRIPTION OF THE DRAWINGS

FIG. 1 shows a diagram of a FlipTrap vector. SA: splice acceptor sequence; SD: splice donor sequence; pA: polyadenylation signal; dark triangles: site specific recombination sites in opposite orientations; light triangles: site specific recombination sites in opposite orientations different from those used for the dark triangles.

FIG. 2 shows the design of a FlipTrap construct using a uni-directional site-specific recombinase. Symbols and abbreviations used are consistent with those shown in FIG. 1. RS-A: represents the first recombination site. RS-B: represents the second recombination site.

FIG. 3 shows the FlipTrap vector, pT2kdelta-FlipTrap.

### BRIEF DESCRIPTION OF THE SEQUENCES

SEQ ID NO: 1 nucleic acid sequence of pT2kdelta-FlipTrap vector.

SEQ ID NO: 2 splice acceptor (SA) sequence of pT2kdelta-FlipTrap from zebrafish.

SEQ ID NO: 3 splice donor (SD) sequence of pT2kdelta-FlipTrap from zebrafish.

SEQ ID NO: 4 loxP recombinase site of pT2kdelta-FlipTrap vector.

SEQ ID NO: 5 loxPV recombinase site of pT2kdelta-FlipTrap vector.

SEQ ID NO: 6 Tol2 transposon of pT2kdelta-FlipTrap vector.

### DETAILED DESCRIPTION

The invention provides vectors and methods for generating genetic alleles that in the initial conformation make a fusion protein with a given marker and after conditional recombination create a mutant allele with a second marker. This approach is called FlipTrapping. This method has a number of useful properties. It allows researchers to monitor the expression and localization of endogenous proteins through the use of a marker fusion protein. It also permits conditional mutagenesis in a wide range of plants and animals to identify gene function. Additionally, the conversion from a functional to a nonfunctional allele can be monitored using two different markers (a first detectable marker referred to as Marker1, and a second detectable marker referred to as Marker2).

The present disclosure provides a nucleic acid vector that is suitable for use in FlipTrapping. In one embodiment, a nucleic acid vector of the present disclosure has a first and second vector sequence that allows for either random or sequence specific integration into a chromosome. Said vector has a first pair of recombination sites, which can be recognized by a sequence-specific recombinase, and are in opposite orientations.

A vector of the present disclosure also has a second pair of recombination sites which are in opposite orientations to one another. The second pair of recombination sites and the first pair are different sequences. The recombination sites can be recognized by the same recombinase or by different recombinases. The second pair of recombination sites flank a second marker protein that has a polyadenylation site. The second pair also flanks one of the recombination sites of the first pair of recombination sites. A vector of the present disclosure also has a splice acceptor sequence and a splice donor sequence. The splice donor sequence is flanked by the first recombination sites, but not by the second pair of recombination sites.

According to the methods of the present disclosure, a cell or organism is put in contact with (e.g. transformed) with a vector as described herein, such that the vector integrates into the cell's DNA. A cell or organism put in contact with a vector of the present disclosure is the receiving cell or receiving organism, respectively. In one embodiment, the vector integrates into the cell's genomic DNA. It is preferred that the receiving cell or receiving organism is able to express one or more recombinases in order to induce recombination of the first and second pairs of recombination sites.

To establish FlipTrap cell lines or organisms, a FlipTrap vector, also called a FlipTrap cassette, is integrated into the genome either randomly or site specifically. If a FlipTrap cassette integrates into an intron in the same orientation as the endogenous gene, then splicing will occur between the upstream endogenous exon and the cassette's splice acceptor (SA) and between the cassette's splice donor (SD) and the downstream endogenous exon generating a transcript encoding a fusion protein. Such an allele is called a “Fusion Trap” because it traps the splicing signals of an endogenous gene to generate a fusion protein if the construct is in the correct orientation and in-frame. Upon addition of the site-specific recombinase(s) that recognize the two sets of recombination sites, recombination will occur such that Marker1 and the SD will be deleted and Marker2 and the poly-A (pA) sequence will now be in the sense orientation with respect to the endogenous gene. This conformation is called a “Gene Trap” because it traps the upstream exon, but then terminates the transcript prematurely because of the pA sequence.

Basic Use

The FlipTrap strategy is useful for a number of reasons, principally because it produces a conditional mutant allele. In the initial conformation (Fusion trap), the marker will only be expressed if it is in-frame with the endogenous protein and is properly spliced to make a fusion protein. Because of the need to make a functional fusion protein, care should be taken that the artificial exon created by the FlipTrap construct is evenly divisible by three (i.e. the exon is symmetric). The artificial exon must be symmetric but it can be in any of the three frames so that it can form a fusion trap with all 3 phases of exons. The fusion proteins produced by the present disclosure will often retain their activity meaning that the Fusion trap allele is functional. This functional fusion trap allele can be converted into a mutant gene trap allele by addition of the site specific recombinase(s). Importantly, the transition from the fusion trap conformation to the gene trap conformation can be monitored using Marker1 and Marker2, which is a significant advantage over the approaches currently available for making conditional alleles.

Markers

According to the method and vector of the present disclosure, any marker can be used for Marker1 (a first detectable marker) and Marker2 (a second detectable marker) as long as it is selectable or visible. If the FlipTrap cassette cannot be inserted efficiently into the genome, then a detectable marker can be used to select or observe rare cases in which the FlipTrap cassette has inserted into an intron to form a fusion protein. Suitable detectable markers can be one of many. In one embodiment a detectable marker is a selectable marker. In one embodiment a selectable marker is an antibiotic resistance gene. In one embodiment, Marker1 is an antibiotic resistance gene. In one embodiment, Marker1 is an antibiotic resistance gene that is the neomycin resistance gene In one embodiment, Marker 2 is an antibiotic resistance gene. In another embodiment, Marker2 is an antibiotic resistance gene that is the neomycin resistance gene. An antibiotic resistance gene used in a vector according to the present disclosure, can be any antibiotic resistance gene known in the art.

Visible markers are beneficial because they allow FlipTrap alleles to be identified visually and allow the expression of the fusion protein to be monitored visually. In one embodiment, a detectable marker is a visible marker. In one embodiment, Marker1 is a visible marker. In one embodiment, Marker1 is a visible marker that is an enzyme that can be used with chromogenic substrates. In one embodiment, Marker1 is beta-galactosidase. Particularly useful for visible markers are fluorescent proteins such as Green Fluorescent Protein (GFP) because they can be visualized in intact cells or organisms without the addition of exogenous substrates. In one embodiment of the present disclosure, two different markers are used for Marker1 and Marker2 so that they can be distinguished. In another embodiment, the two different markers are two different fluorescent proteins with different spectral properties. In one embodiment, Marker1 is GFP. In another embodiment, Marker2 is GFP. In one embodiment, Marker1 is a yellow fluorescent protein. In one embodiment, Marker1 is a red fluorescent protein. In another embodiment, Marker2 is a yellow fluorescent protein. In another embodiment, Marker2 is a red fluorescent protein. In another embodiment, Citrine (yellow) and Cherry (red) fluorescent proteins are used as Marker1 and Marker2, respectively.

Care should also be taken to minimize the length of Marker1. There is a bias against proper splicing of large internal exons. If Marker1 is too long, it may not be properly spliced. Care should also be taken so that Marker1 does not contain internal “cryptic” SA, SD, or pA sequences. Such sites can be identified using consensus and computer based prediction algorithms such as SpliceView or SpliceSiteFinder. (Shapiro and Senapathy (1987), Nucleic Acids Res., v 15, n 17, pp 7155-7174; Senapathy et al (1990), Methods Enzymol., vol. 183, p. 252; http://www.genet.sickkids.on.cakali/splicesitefinder.html)

Potential cryptic splice and polyA sites in Marker1 should be removed by silent site-specific mutagenesis. Potential cryptic splice sites and polyA sites should also be avoided in the antisense strand of pA and Marker2 to ensure proper recognition and splicing of the markers artificial exon. Additionally, markers should not have a stop codon so that an internal fusion protein can be formed. It is not necessary for Marker1 to have a start codon either, since it will form an internal fusion protein. The size of Marker2 is not as critical since there is less bias toward large terminal exons compared with internal exons. For this reason, a stop codon and an internal ribosome entry site (IRES) sequence can be used in front of Marker2 so that the trapped protein is truncated and Marker2 is expressed in the gene trap conformation. Additional nucleic acid insertion sequences may be included in a FlipTrap vector according to the present invention.

In an alternative embodiment, a vector of the present invention can be constructed using the IRES sequence of the FMDV (foot and mouth disease virus) or EMCV (encephalomyocarditis virus) cleavage factor 2a, these are also referred to as viral sequences (Chinnasamy, D. et al., (2006), Virol J. Vol. 3, p. 14; Furler, S., et al., (2001) Gene Ther. Vol. 8, 864-873).

Additionally, a vector according to the present invention may comprise a viral LTR (long terminal repeat) (Bushman, F. D. (2003), Cell, Vol. 5, 135-138).

In another embodiment, the method and vector of the present disclosure does not contain any marker. In other words, the vector of the present invention does not contain Marker1 or Marker2.

Recombinases

In one embodiment, any enzyme that causes recombination between two sites based on a specific sequence can be used for the recombinases in the FlipTrap vector of the present disclosure. The specific recombination site should be of sufficient length so that it is rare or absent in the genome of the receiving cell or receiving organism. In another embodiment, the recombinases used in the FlipTrap vector of the present disclosure include, but are not limited to the Cre and FLP recombinases. (FlpE: Rodriguez, C. I., Buchholz, F., Galloway, J., Sequerra, R., Kasper, J., Ayala, R., Stewart, A. F. & Dymecki, S. M. (2000) Nat. Genet. 25, 139-140.) Recombinases of the present invention can be introduced into a receiving cell or organism via any of the several methods know in the art. Such introduction can be carried out via transfection, injection, transduction, infection, transformation and the like as is known to one of skill in the art.

In one embodiment, two different recombinases can be used along with their respective two cognate recombination sites. In another embodiment a single recombinase is used for two different pairs of heterotypic sites. For example, the Cre recombinase can be used along with one pair of loxP (SEQ ID NO: 4) sites and one pair of loxPV (SEQ ID NO: 5) sites. Alternatively one pair of lox511 sites (WO 2006056617-A) could be used in place of either pair of loxP or loxPV sites. The advantage of using a single recombinase with two pairs of heterotypic sites is that only a single recombinase needs to be provided to cause recombination.

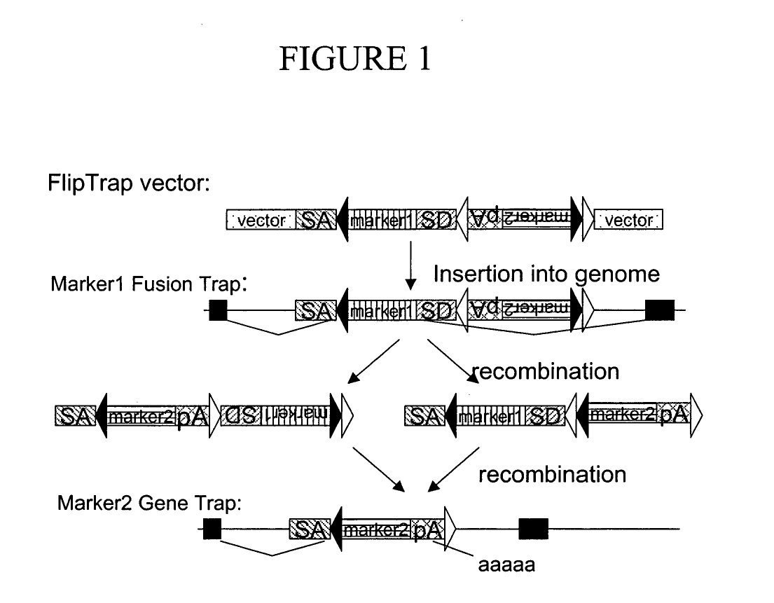

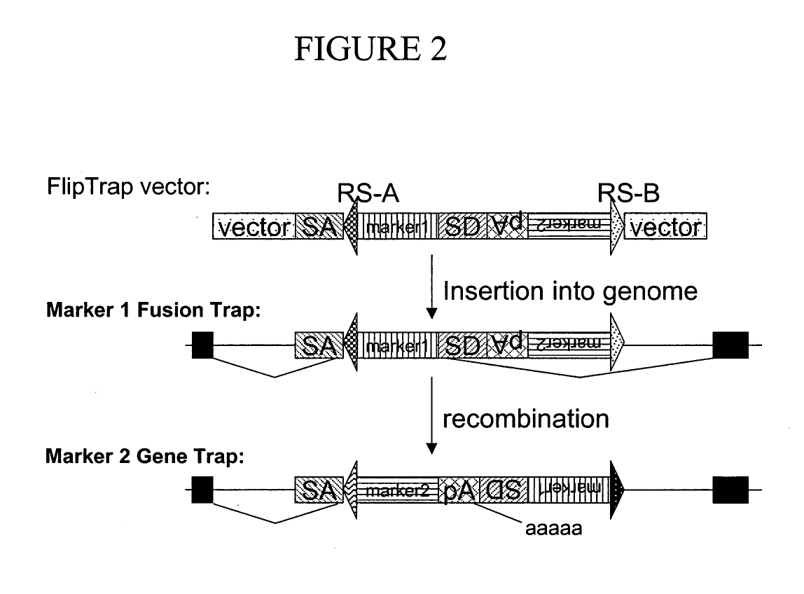

The position of the recombination sites in the construct is somewhat flexible, but the relative order and orientation of the sites should be as indicated in FIGS. 1 and 2. Changing the position of the recombination sites will change what parts of the construct are flipped and deleted. The recombination sites can be moved as long as Marker2-pA is brought into proper position after recombination.

In one embodiment, the recombinase is provided genetically, such the recombinase is encoded genomically in the cell. In another embodiment, the recombinase is expressed from an inducible promoter in a transgenic cell/organism to be used in conditional mutagenesis. In another embodiment, the recombinase is expressed in a tissue-specific manner in a transgenic cell/organism to be used in conditional mutagenesis.

Vectors

The FlipTrap cassette can be integrated into the genome in a number of ways. In one embodiment a method is used that ensures only a single copy of the cassette is inserted at each site (rather than a concatamer). In one embodiment retroviruses are used to insert the FlipTrap cassette randomly throughout the genome. In another embodiment a transposon-based vector such as Tol2 (SEQ ID NO: 6) is used to insert the FlipTrap cassette throughout the genome. One of skill in the art can use similar transposon-based vectors, e.g. Sleeping Beauty (U.S. Pat. No. 6,613,752). Since transposon-based vectors require minimal extraneous sequence to be added to the vector, these can work with high efficiency, and can target a very large number of sites in the genome. According to one embodiment, the nucleic acid backbone of a FlipTrap vector according to the present invention is a plasmid.

Splice Acceptor, Splice Donor and Poly-A Sequences

The choice of the SA, SD, and pA sequences is important to ensure proper splicing. Most naturally occurring internal exons are much smaller than the likely size of Marker1. A typical exon is 100-200 bp long where as Marker1 will likely be >700 bp. There are, however, naturally occurring internal exons that are as large as the potential size of Marker1. In one embodiment, the sequences for SA and SD, are those of the splice acceptor and donor from a naturally occurring large internal exon from the same species as the receiving cell or organism. Such large internal exons can be identified through sequence analysis in organisms with a substantial genomic sequence database.

In one embodiment, the SA sequence includes ˜300 bp upstream of the intron-exon boundary and ˜15 bp downstream of the intron-exon boundary. These flanking sequences added to the SA sequence can help ensure the proper splicing signals are included once the vector has integrated. In one embodiment, the SD sequence includes ˜15 bp upstream of the exon-intron boundary and ˜300 bp downstream of the exon-intron boundary. These selected sequences can be analyzed using a splicing prediction algorithm to ensure that the intended splice sites are very strong and there are no other strong splice sites in the SA or SD. The frame of the ˜15 bp exonic regions at the end of the SA and beginning of the SD do not necessarily need to match the frame of Marker1, but it is important that these sequences do not contain any stop codons. It is also useful for the ˜15 bp exonic regions to encode a stretch of amino acids that forms a good linker between the trapped protein and Marker1. Amino acids that make good linkers are those which are small and not charged.

SpliceView or SpliceSiteFinder, (Shapiro and Senapathy (1987), Nucleic Acids Res., v 15, n 17, pp 7155-7174; Senapathy et al (1990), Methods Enzymol., vol. 183, p. 252; http://www.genet.sickkids.on.cakali/splicesitefinder.html) are helpful resources for designing a SA and/or SD sequence to be used according to the present invention.

In one embodiment, a pA is chosen that contains a poly-A signal only in the sense direction so that the transcript is terminated and polyadenylated only in the gene trap conformation and not in the fusion trap conformation. In one embodiment, a poly-adenylation (poly-A; pA) sequence is incorporated into a FlipTrap vector of the present disclosure such that it is only in the sense direction. Computer programs can also be used to analyze potential pA sequences. Such pA sequences can be identified through sequence analysis in organisms with a substantial genomic sequence database by mapping where cDNA sequences end and identifying pA site consensus signals. A region spanning from just past the stop codon through the site of polyadneylation to ˜200 bp past the site of polyandenylation can be used for a pA.

Choice of biological systems

A FlipTrap vector and/or method can be used in any organism or cell line that has introns. Established genetic organism systems for in vivo animal cell analysis in an intact animal, includes but is not limited to C. elegans, Drosophila melanogaster, zebrafish, medaka (rice fish), and mouse. In these systems, it is easier to “homozygose” (make homozygous) the conditional alleles in order to investigate possible recessive phenotypes. Biological systems that can be imaged well are appropriate if visible markers are used. Cell lines can also be used such as any animal or plant cell. Cultured animal cell lines such as embryonic stem (ES) cells can be used as the receiving cell. Mouse ES cells are an example of an established cultured cell line. Additionally, fertile animals can be recreated from mouse ES cells. Though making mutant alleles homozygous in mutant alleles in cell lines is more difficult than in sexual organisms, cell lines are possible receiving cells for a FlipTrap vector as disclosed (Vazquez, J C et al., (1998) Transgenic Res. Vol. 7, 181-193; Araki, K et al., (1997) J. Biochem Vol. 122, 977-982).

Identifying FlipTraps

FlipTrap alleles can be created in a directed manner, for example, by gene targeting using homologous recombination. In one embodiment, FlipTrap alleles are integrated into the genome of a receiving cell or organism by homologous recombination of a specific gene or genes. This approach is appropriate in systems with established gene targeting techniques such as mouse ES cells. (Floss, T. & Wurst, W. (2002) Methods Mol. Biol. vol 185, 347-379; Mansouri, A. (2001) Methods Mol. Biol. vol. 175, 397-413; Araki, K. et al., (1995) PNAS, vol. 92, 160-164; Sakai, K & Miyazaki, J. (1997) Biochem Biophys Res Commun. Vol. 237, 318-324).

FlipTrap alleles can also be identified through screening after random integration of the FlipTrap construct into the genome of a receiving cell or organism. In one embodiment, a FlipTrap vector of the present disclosure is integrated into the genome of a receiving cell or organism by random integration. This approach can be used in any system with established techniques for random integration of a DNA cassette. The use of transposon-based vectors makes this approach possible in many organisms. Screening can be done by visually screening individuals for expression of a visible marker. Selection can also be used to identify FlipTraps if a selectable marker is used in systems that have established selectable marker techniques such as mouse ES cells. (Vazquez, J C et al., (1998) Transgenic Res. Vol. 7, 181-193; Araki, K. et al., PNAS, Vol. 92, 160-164).

Selecting for the presence of the first (Marker1) or second (Marker2) selectable marker sequence comprises selecting for the presence of the protein expressed from the marker gene. If the selectable marker gene is an antibiotic resistance gene, then selection of this marker will be to look for growth of the cell or organism in the presence of the specific antibiotic. If the selectable marker is a visible marker, it will be necessary to observe the marker (e.g. beta-galactosidase or fluorescence) by a method that corresponds to the receiving cell or organism.

Use of Uni-Directional Site-Specific Recombinases

An alternative to the above recombination approach that uses a unidirectional recombinase is shown in the schematic in FIG. 2. A recombinase that catalyzes the recombination of two sites (RS-A and RS-B) in a unidirectional manner can be used instead of two pairs of heterotypic sites. An appropriate recombinase for this approach should have recombination sites that are rare or nonexistent in the genome of the receiving cell or organism. The two recombination sites should be recombined by the recombinase to generate a product that is not a substrate for further recombination (i.e. it is uni-directional). An appropriate uni-directional recombinase is the integrase from phi C31.

### EXAMPLES

pT2Kdelta-FlipTrap

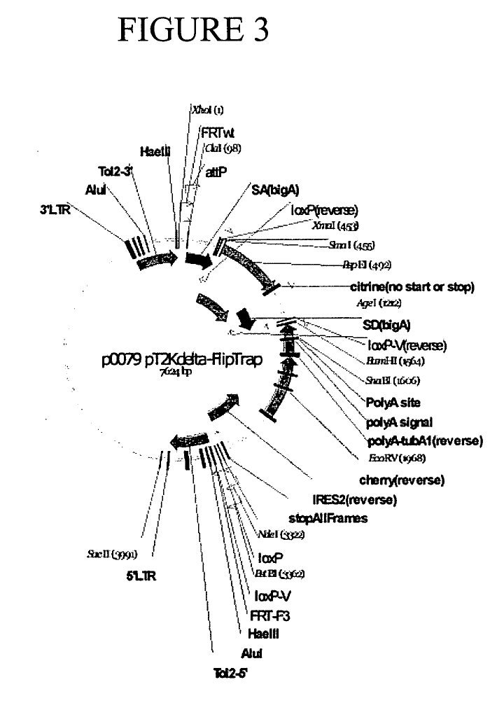

FIG. 3 shows a diagram of a flip trap vector that has been used successfully (pT2Kdelta-FlipTrap). This vector uses the Tol2 transposon (SEQ ID NO: 6) to permit random, efficient insertion into the genome of zebrafish and many other species. Alternatively, the Sleeping Beauty transposon sequence can be used (U.S. Pat. No. 6,613,752). The vector uses a SA (SEQ ID NO: 2) and SD (SEQ ID NO: 3) from a naturally occurring large internal exon in zebrafish. The sequences were found by computationally searching all annotated zebrafish genomic sequence to make a list of all internal exons along with their size and the sizes of their 5′ and 3′ introns. Since it is easier to splice in a large exon if it is surrounded by small introns, entries on the list that contain a small (<900 bp) 5′ or 3′ intron were eliminated. This list was sorted such that the largest internal exons were at the top. Each of the top 20 sized internal exons were used to perform BLAST searches of available cDNA and EST databases to find corresponding sequences. Entries on the top 20 list that did not contain strong evidence from sequenced cDNAs for constitutive splicing across both the 5′ and 3′ exon junctions were eliminated. Pairs of potential SA and SD sequences were made for each of the remaining exons on the list by taking from 300 bp upstream of the intron-exon junction to 15 bp downstream of the intron-exon junction for the SA and from 15 bp upstream of the exon-intron junction to 300 bp downstream of the exon-intron junction for the SD. Pairs of SA and SD sites that contained repetitive sequences were eliminated. The remaining SA and SD sites were scored using SpliceSiteFinder. Pairs of SA and SD sites in which splicing were incorrectly predicted were eliminated. The 15 bp of exonic sequence in both the SA and SD for all pairs were then virtually translated in all 3 frames since these will form a linker between Marker1 and the trapped protein. Pairs containing stop codons or an abundance of bulky or charged amino acids were eliminated. A pair of SA and SD was chosen from those that remained on the list based on the best splice score and the best amino acids to form a linker. This SA and SD sites were PCR amplified from genomic DNA and correspond to SEQ ID NO: 2 and SEQ ID NO: 3 presented here. This pair of SA and SD sequences comes from a large internal exon flanked by large introns for a naturally occurring gene in zebrafish that has not been studied much to date.

Two fluorescent proteins were used as visible detectable markers. Citrine, a yellow fluorescent protein, was used as Marker1 and mCherry, a red fluorescent protein was used as Marker2 (Baird, G S et al., (2000) PNAS, vol. 97, 11984-11989; Heikal, A A et al., (2000) PNAS, vol. 97, 11996-12001). The poly-adenylation (pA) site is from zebrafish and does not contain any splice or poly-adenylation signals in the reverse orientation.

A FlipTrap Vector in vivo

The pT2Kdelta-FlipTrap vector (SEQ ID NO: 1) has been used successfully in zebrafish. More specifically, 100 zebrafish fertilized eggs with pT2Kdelta-FlipTrap DNA at a concentration of 40ng/ul along with RNA encoding the Tol2 transposase also at a concentration of 40ng/ul. Embryos were screened at 1, 2, and 3 days post-fertilization. Of the 100 injected fish, ten showed yellow fluorescence in a subset of cells, and six of the ten showed a noticeable sub-cellular distribution of yellow fluorescent due to a functional citrine fusion protein being formed.

Gene trapping using the FlipTrap vector as disclosed herein, has been observed in a wide range of cells including muscle, neurons, epidermis, notochord sheath, and mesenchyme.

In a separate series of experiments, 500 zebrafish fertilized eggs were injected with pT2Kdelta-FlipTrap vector DNA at a concentration of 40ng/ul along with RNA encoding the Tol2 transposase also at a concentration of 40ng/ul. The eggs were raised to adulthood. The adults were intercrossed and the resulting F1 embryos were screened for fluorescence. 46 independent lines of fluorescent FlipTrap zebrafish were observed. The positive F1 embryos were raised to establish FlipTrap lines in the fusion conformation. Three lines of these F1 fish were crossed to generate F2 eggs which were injected with Cre recombinase RNA at a concentration of 0.2ng/ul. These embryos showed a loss of yellow fluorescence and a corresponding gain in red fluorescence showing that flipping occurred. The trapped gene has been cloned in 10 of these lines using 3′RACE and shows that proper fusion transcripts are formed by the pT2Kdelta-FlipTrap vector.

In summary, gene trapping provides insertional mutagenesis strategies for monitoring the expression and localization of endogenous proteins. In accordance with the disclosure herein, a gene trapping vector (FlipTrap) and a method of fabrication and use thereof, are disclosed which provide for an efficient means of trapping and analyzing a gene or genes of interest in a cell or organism.

While illustrative embodiments have been shown and described in the above description, numerous variations and alternative embodiments will occur to those skilled in the art. Such variations and alternative embodiments are contemplated, and can be made without departing from the scope of the invention as defined in the appended claims.

1. A nucleic acid vector comprising:
a nucleic acid backbone sequence;
a splice acceptor sequence;
a splice donor sequence;
a first recombination site and a second recombination site forming a first recombination site pair capable of being recognized by a first recombinase, wherein the first and second recombination sites are in opposite orientations;
a third recombination site and a fourth recombination site forming a second recombination site pair capable of being recognized by the first recombinase or a second recombinase, wherein the first and second recombination sites are in opposite orientations, and wherein the second recombination site pair flanks the second recombination site, but does not flank the first recombination site; and
a polyadenylation sequence.

2. The nucleic acid vector of claim 1, further comprising a first detectable marker sequence.

3. The nucleic acid vector of claim 2, further comprising a second detectable marker sequence.

4. The nucleic acid vector of claim 1, further comprising a nucleic acid insertion sequence

5. The nucleic acid vector of claim 4, wherein the nucleic acid insertion sequence is a viral sequence, a transposon sequence, or a homologous recombination sequence.

6. The nucleic acid vector of claim 5, wherein the nucleic acid insertion sequence is a Tol2 (SEQ ID NO: 6) transposon.

7. The nucleic acid vector of claim 5, wherein the nucleic acid insertion sequence is a viral LTR.

8. The nucleic acid vector of claim 1, wherein the nucleic acid backbone is a plasmid.

9. The nucleic acid vector of claim 1, wherein the splice acceptor sequence is: SEQ ID NO: 2.

10. The nucleic acid vector of claim 1, wherein the splice donor sequence is: SEQ ID NO: 3.

11. The nucleic acid vector of claim 1, wherein the first and second recombination sites are selected from SEQ ID NO: 4 and 5.

12. The nucleic acid vector of claim 1, wherein the first recombinase and the second recombinase are the same and the first recombination site pair is heterotypic with respect to the second recombination site pair.

13. The nucleic acid vector of claim 1, wherein the first recombinase is Cre.

14. The nucleic acid vector of claim 1, wherein the first recombinase is Flp.

15. The nucleic acid vector of claim 2, wherein the first detectable marker sequence encodes a selectable marker.

16. The nucleic acid vector of claim 15, wherein the selectable marker is an antibiotic resistance gene.

17. The nucleic acid vector of claim 16, wherein the antibiotic resistance gene is a neomycin resistance gene.

18. The nucleic acid vector of claim 1, wherein the first detectable marker sequence encodes a visible marker.

19. The nucleic acid vector of claim 18, wherein the visible marker is a fluorescent protein.

20. The nucleic acid vector of claim 19, wherein the fluorescent protein is green fluorescent protein.

21. The nucleic acid vector of claim 18, wherein the visible marker is a chromogenic enzyme.

22. The nucleic acid vector of claim 21, wherein the chromogenic enzyme is beta-galactosidase.

23. The nucleic acid vector of claim 1, wherein the polyadenylation sequence is not effective in the reverse orientation.

24. A method of creating a conditional allele, the method comprising:
introducing into a cell, a vector of claim 1;
introducing into the cell, a first recombinase creating a first recombination event and a second recombination event.

25. The method of claim 24, further comprising introducing into the cell a second recombinase, wherein the second recombinase creates the second recombination event.

26. A method of creating a conditional allele, the method comprising:
introducing into a cell, a vector of claim 3;
introducing into the cell, a first recombinase, wherein the first recombinase creates a first recombination event;
introducing into the cell, a second recombinase, wherein the second recombinase creates a second recombination event;
selecting for the presence of a protein expressed from the first detectable marker;
selecting for the presence of a protein expressed from the second detectable marker.

27. The method of claim 26, wherein the first and second recombinases are introduced by transfection.

28. The method of claim 26, wherein the first and second recombinases are encoded genomically in the cell.

29. The method of claim 26, wherein the first and second recombinases can be expressed from an inducible promoter.

30. The method of claim 26, wherein the first and second recombinases can be expressed in a tissue-specific fashion.

31. The method of claim 24, wherein the cell is an animal cell or a plant cell.

32. The method of claim 31, wherein the animal cell is a cultured cell.

33. The method of claim 32, wherein the cultured cell is an embryonic stem cell.

34. The method of claim 33, wherein the embryonic stem cell is a mouse embryonic stem cell.

35. The method of claim 31, wherein the animal cell is in vivo, in an intact animal.


SEQUENCE LISTING (6 sequences)

SEQ ID NO: 1 - DNA, 7624 bp, Artificial Sequence
  Description of Artificial Sequence Synthetic construct
tcgaggaagttcctattctctagaaagtataggaacttcactagtctacgcccccaactgagagaactcaaaggttaccccagttggggcactacatcgattcaggaacctcacagactgcaggttgagctccccctgaagggctcactaacaccctttttttcacaacaaccaagtttttttcatgtggtcttccatccaggtactaaccaggcttagccctgcttagcttcagtgagtaaccagtcttgggcagcaagttgatttggctgtaccaataatgatttttgtaataataatttttgcaattagaatgctactgattgagatattacacactccaatttgatctgaaactaaaagtttctgttgtcattacagaggaaattcctgactcttttgataatgtaaatgtgctgtgttgcaggacgaactggtcgctacacattcacccgggataacttcgtatagcatacattatacgaagttatccggagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagaccggtccttcaaacattgggctacaggtgagtgtacctgaagaaagaacacaatttacttacacttacatttcaaacagataacttgacttagattgcttttagttttttggtgtggactaagtccatttgacattgaggttaatttaattggtgtgaaagttttcgccaaactcaggcacataacagaaagctaaaaacccaattgttatgaaagcggctagtcctaaatcaatgagtggattaagaaattcacacagaagaaacctttttaaaacgcctagaaacctggtagctaggtcatataactgagttcctgaggtgaaagccccttattgtaaactacccaaagggatccataacttcgtataggatactttatacgaagttattacgtaactaatctacttttccttcatcccaagtggttggaagccaaacctgccaaagattagtaagcagattacttatcttcaatgcccagaatgtcttaaaaccaaaagcttatgttaacatgtacaagaaagatttttaaagaactgcacatggacagattgtagcacatcaccatcaatttattccaaatttggcaagcaaattccacaaataataagcatactggccataacaacacaacaactgatcaagaatgtgtctctaacatttaggagtagactttacacttgatacctcatgtttatcgtgtgtaaattagcggtctactgtgttcagttttagacatttagtggatattgatatcttacttgtacagctcgtccatgccgccggtggagtggcggccctcggcgcgttcgtactgttccacgatggtgtagtcctcgttgtgggaggtgatgtccaacttgatgttgacgttgtaggcgccgggcagctgcacgggcttcttggccttgtaggtggtcttgacctcagcgtcgtagtggccgccgtccttcagcttgagcctctgcttgatctcgcccttcagggcgccgtcctcggggtacatccgctcggaggaggcctcccagcccatggtcttcttctgcattacggggccgtcggaggggaagttggtgccgcgcagcttcaccttgtagatgaactcgccgtcctggagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcaccatggttgtggccatattatcatcgtgtttttcaaaggaaaaccacgtccccgtggttcggggggcctagacgtttttttaacctcgactaaacacatgtaaagcatgtgtaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggctcagtcagctatctagataacttcgtataatgtatgctatacgaagttatcatatgataacttcgtataaagtatcctatacgaagttatttcgaagaagttcctattcttcaaatagtataggaacttcagatcaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattactcaagtactttacacctctggttcttgaccccctaccttcagcaagcccagcagatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacccacaatgacggctactacatggtgccattccttcctctttataggaatggagactacctcctgtccaacaaggctcttggatacgagtacgcctacctgttggacccaggtcattgcacaacaccagaaatgccctctgatctgcaaaagacgtgaatatctgttcagacacccatatccactctgttccacacaggtcagaggtttgtccaggagttcttgacagaggtgtaaaaagtactcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccc

SEQ ID NO: 2 - DNA, 348 bp, Brachydanio rerio
tcaggaacctcacagactgcaggttgagctccccctgaagggctcactaacaccctttttttcacaacaaccaagtttttttcatgtggtcttccatccaggtactaaccaggcttagccctgcttagcttcagtgagtaaccagtcttgggcagcaagttgatttggctgtaccaataatgatttttgtaataataatttttgcaattagaatgctactgattgagatattacacactccaatttgatctgaaactaaaagtttctgttgtcattacagaggaaattcctgactcttttgataatgtaaatgtgctgtgttgcaggacgaactggtcgctacacatt

SEQ ID NO: 3 - DNA, 344 bp, Brachydanio rerio
ttcaaacattgggctacaggtgagtgtacctgaagaaagaacacaatttacttacacttacatttcaaacagataacttgacttagattgcttttagttttttggtgtggactaagtccatttgacattgaggttaatttaattggtgtgaaagttttcgccaaactcaggcacataacagaaagctaaaaacccaattgttatgaaagcggctagtcctaaatcaatgagtggattaagaaattcacacagaagaaacctttttaaaacgcctagaaacctggtagctaggtcatataactgagttcctgaggtgaaagccccttattgtaaactacccaaag

SEQ ID NO: 4 - DNA, 34 bp, Artificial Sequence
  Description of Artificial Sequence Synthetic construct
ataacttcgtataatgtatgctatacgaagttat

SEQ ID NO: 5 - DNA, 34 bp, Artificial Sequence
  Description of Artificial Sequence Synthetic construct
ataacttcgtataaagtatcctatacgaagttat

SEQ ID NO: 6 - DNA, 1027 bp, Artificial Sequence
  Description of Artificial Sequence Synthetic construct
ctcaaaaattttactcaagtgaaagtacaagtacttagggaaaattttactcaattaaaagtaaaagtatctggctagaatcttacttgagtaaaagtaaaaaagtactccattaaaattgtacttgagtattaaggaagtaaaagtaaaagcaagaaagaaaactagagattcttgtttaagcttttaatctcaaaaaacattaaatgaaatgcatacaaggttttatcctgctttagaactgtttgtatttaattatcaaactataagacagacaatctaatgccagtacacgctactcaaagttgtaaaacctcagatttaacttcagtagaagctgattctcaaaattgttagtgtcaagcctagctcttttggggctgaaaagcaatcctgcagtgctgaaaagcctctcacaggcagccgatgcgggaagaggtgtattagtcttgatagagaggctgcaaatagcaggaaacgtgagcagagactccctggtgtctgaaacacaggccagatgggccaattcgcgaagatacggccacgggtgctcttgatcctgtggctgattttggactgtgctgctcgcagctgctgatgaatcacatacttcctccattttcttccactgattgactgttataatttccctaatttccaggtcaaggtgctgtgcattgtggtaatagatgtgacatgacgtcacttccaaaggaccaatgaacatgtctgaccaatttcatataatgtgaaaacgattttcataggcagaataaataacatttaaattaaactgggcatcagcgcaattcaattggtttggtaatagcaagggaaaatagaatgaagtgatctccaaaaaataagtactttttgactgtaaataaaattgtaaggagtaaaaagtacttttttttctaaaaaaatgtaattaagtaaaagtaaaagtattgatttttaattgtactcaagtaaagtaaaaatccccaaaaataatacttaagtacagtaatcaagtaaaattact